(19) United States
(12) Patent Application Publication
(10) Pub. No.: US 2016/0066809 A1
(43) Pub. Date: Mar. 10, 2016

(54) Elastic garment for positioning and fixing ECG electrodes

(71) Applicants: Zhiyuan Luo, Shanghai (CN); Yongxin Sun, Orlando, FL (US)

(72) Inventors: Zhiyuan Luo, Shanghai (CN); Yongxin Sun, Orlando, FL (US)

(21) Appl. No.: 14/872,141

(22) Filed: Oct. 1, 2015

(51) Int. Cl. A61B 5/0408; A61B 5/04; A61B 5/00

(52) U.S. Cl. CPC A61B 5/04085; A61B 5/6805; A61B 5/04012; A61B 5/0006

(57) ABSTRACT

An elastic garment provides an easy, quick, user-friendly way of positioning and fixing ECG electrodes. The elastic textile can offset the impact of body shape on the position of ECG electrodes. The elasticity ensures good electrode abutment and prevent electrode dislocation. The garment has labels indicating the significant bony landmarks, which help user wear the garment correctly thus ensuring the position of the electrodes. Optional features include: labels to indicate the position of electrodes; embedded wires, electrodes and connectors to external ECG device. The garment makes it possible for non-professionals to perform ECG examination including the standard 12-lead ECG. It also minimizes electrode dislocation and uncomfortable feelings of wearing ECG device, especially for those under long-term ECG monitoring. It can also help avoid the inconvenient procedure of undressing a patient during ECG examination.

### BACKGROUND OF THE INVENTION

(1) Field of the Invention

The present invention relates to an ECG electrode wearable, and more particularly to an elastic ECG electrode wearable garment that enables accurate positioning and fixing of ECG electrodes.

(2) Description of Related Art

The standard 12-lead ECG consists of 3 limb leads (leads I, II, and III), 3 augmented limb leads (leads aVR, aVL, and aVF), and 6 precordial leads (V1 through V6). However, the traditional standard lead ECG does not actually require 12 electrodes, but 10 electrodes. Six chest electrodes are placed on the following locations: V1, fourth intercostal space at the right sternal border; V2, fourth intercostal space at the left sternal border; V3, midway between V2 and V4; V4, fifth intercostal space in the midclavicular line; V5, in the horizontal plane of V4 at the anterior axillary line, or if the anterior axillary line is ambiguous, midway between V4 and V6; and V6, in the horizontal plane of V4 at the midaxillary line. Four standard “limb” electrodes are placed on or near each of the four appendages (RA, LA, RL, and LL, for the right and left arms and legs, respectively). To reduce the noise from motion of the arms and legs during ambulatory and exercise electrocardiography, modified electrode placement schemes like Mason-Likar lead placement have also gained clinical acceptance, in which the arm electrodes are placed in the infraclavicular fossae medial to the deltoid insertions and the left leg electrode is placed midway between the costal margin and iliac crest in the left anterior axillary line.

Conventional clinical ECG is quite complex. The operator has to manually position and fix the electrodes at the proper sites on the skin. Inaccurate placements will result in misleading recordings which will increase the complexity of analysis and result in misdiagnosis. Accurate placement of electrodes requires extensive training and expertise which is beyond the skills possessed by the untrained people, making self-administration impossible.

The conventional ECG electrodes are differentiated only by color and labels. The operator must be careful to connect lead wires correctly at both ends to avoid a faulty recording. Lead wires are easily to get tangled. They must be kept trim and orderly, so as to reduce motion artifact and noise and eliminate the possibility of accidental dislocation.

The electrodes of the conventional ECG have chance to get dislocated and cause deviation in ECG recordings. During the application of conventional ECG, especially if the patient is uncooperative or unrestful, there are chances for electrode dislocation. This is frequently seen in children, psychotic patients or patients in transportation, and it is one of the key reasons why conventional ECG is not suitable for constant monitoring.

Administration of ECG requires patients to expose the precordial area, which sometimes makes the patient physically or psychologically uncomfortable, especially in a cold environment or when repeated ECG examinations are necessary.

Household ECG devices develop rapidly in recent years. Many small, affordable and convenient devices are available in the market. Due to the complexity of standard-lead ECG, most of the available household devices only support simplified ECG which usually uses less than 4 electrodes without accurate requirement for electrode position. These ECG recordings are not appropriate for clinical analysis or diagnosis, but only the reference for limited diseases. Though simplified, the administration procedure of most household ECGs does not make big difference from clinical ECG in many aspects, i.e. they are still not convenient enough. Due to the complexity of administration, most of them are still perceived as ‘clinical’ devices only suitable for the patients and not accepted by the ordinary people as a device for daily cardiac monitoring. Another disadvantage of the household devices is that most of them cannot support constant ECG monitoring.

Many designs have been proposed to solve problems of connecting electrode to the chest. The inventors have designed belts, strips, single-piece device, multiple-piece device, garments or other complex harness to facilitate the attachment of electrodes to the body. These inventions can be basically divided into two categories by their target user.

The first category targets professional users including clinicians, technicians or healthcare workers. These inventions simplified the administration of clinical ECG. However, none of the inventions provided a feasible solution for accurately positioning the electrodes. They still rely heavily on the operators' expertise to decide the electrode position. Almost all these inventions tried to provide one universal device to adapt to all people. To provide the flexibility, these inventions usually have many flexible parts which made them look quite complex, uncomfortable or even weird. Such devices are obviously not suitable for use outside of healthcare facilities. In U.S. Pat. No. 8,954,129 to Schlegel, the inventor aimed to ‘provide a wearable which can be used and applied to numerous individuals without laborious personal customization’. It can be adjusted to fit almost any body shape and might help a lot for professional users. But it is obviously not suitable for ordinary people who want to administrate ECG at home. At the same time, such complex devices are usually difficult to clean and sterilize. In U.S. Pub. No. 20110004088 Grossman proposed an ECG shirt that provided a very humane solution for identifying, specifying and fixing the relative position of the electrodes connected to the patient. The invention tries to take advantage of elastic fabric that can ‘stretch with the enormous variations of body types and sizes’. However, considering that the position of ECG electrodes is not evenly proportional distributed for different body shapes, and that fabric can only ensure proportional extensibility within limited scope, the accuracy of the electrode position cannot be guaranteed if the fabric is dramatically extended. Generally, these inventions still rely highly on the expertise of the operators. None of them provided a practical solution for accurately positioning of ECG electrodes.

The second category targets the ordinary consumers. These inventions are usually more humane and comfortable than the inventions in the first category. U.S. Pat. No. 8,909,318 to Nordstrom depicted an ‘Apparel for physiological telemetry during athletics’. In this design, monitoring components is integrated into a garment, which almost makes no difference from daily clothing. Such a comfortable, aesthetic functional garment is easier to be accepted by consumers. The common disadvantage of these inventions is that they can only monitor very limited simple physical indexes. None of them can follow clinically-accepted ECG standard.

Cardiovascular diseases are usually chronic which commonly start long before people have symptoms. There are many factors that affect cardiovascular health like aging, fatigue, stress, unhealthy life style etc. However, most people don't know much about their cardiovascular health conditions until they encounter first cardiac symptom. It has been proved that Coronary heart disease starts quite early in young subjects. By an autopsy study of 760 young (age 15 to 34 years) victims of accidents, suicides, or homicides, advanced coronary atheromata were seen in 2 percent of men aged 15 to 19. However, coronary heart disease in younger subjects is difficult to establish since it is frequently a silent process. The prevalence of such pathological changes keeps going up with the increase of age. A substantial portion of cardiovascular problems can be detected with ECG before any symptom occurs. A constant monitoring might be especially helpful. In various scenarios where an individual is being monitored, medical assistance may be obtained based on monitored physiological characteristics before a particular health issue becomes fatal.

Constant monitoring is not only helpful for early diagnosis or prevention of fatal health problems, but also helpful for establishing a healthy life style. By constant monitoring, the device can alert early abnormalities. Users can further link the abnormity with their daily activities and avoid the behaviors that have serious impact on their cardiovascular health. However, for a long time, constant ECG monitoring was regarded impractical. There is no generally accepted constant monitoring method. The expense for massive data storage, processing and analysis is also unaffordable for most people.

Things have changed when medicine is combined with internet technology. Cloud storage and computing make massive data processing quite affordable. Health data collection and related services become an overwhelming trend even for the healthy population. People use portable devices to collect health data and synchronize the data to the cloud. In the cloud, the data is saved, processed and analyzed to generate health report for the users. The target user of healthcare devices is gradually shifting from the patients to the normal people who care about their health. It is becoming a new life style in the new era. Among all the health data collectable, ECG is one of the most important health indexes. ECG is also the most suitable data for internet solutions because of the massive data volume and high dependency on data processing capability. From another point of view, constant data collection in healthy population will contribute enormously to the development of cardiovascular science.

To achieve this goal, the fundamental step is to provide a generally acceptable constant ECG monitoring solution.

The object of the present invention is to provide a solution for an electrode wearable for standard or non-standard multi-lead ECG that is friendly to the untrained users, which can help users precisely locate and fix electrode without complex procedures. The second object of present invention is to make ECG wearable psychologically, visually and physically comfortable or even unperceivable after administration, so that such a device is acceptable by both the patient and the healthy population. The third object of present invention is to support continuous ECG monitoring without electrode dislocation on people with basic daily living activities. In short, the ultimate goal of our invention is to make long-term ECG monitoring simple, affordable, comfortable and clinically valuable for most people.

### BRIEF SUMMARY OF THE INVENTION

The present invention relates to an ECG electrode wearable and more particularly to an elastic ECG electrode wearable garment that enables accurate positioning and fixing of the ECG electrodes.

The present applications claims priority to the earlier filed provisional application having Ser. No. 62/182,544, and hereby incorporates subject matter of the provisional application in its entirety.

The present invention aims to provide a simple, quick, affordable, comfortable, aesthetic and clinically valuable device to support standard 12-lead ECG (or other clinical accepted ECG) with continuous monitoring capability. The fundamental goal is to enable ordinary untrained people to administrate ECG without the help of medical professionals. The recorded data is valuable for clinical analysis and diagnosis. It is also valuable for medical organizations especially for continuous ECG monitoring purpose.

The present invention overcomes the disadvantages of the inventions which try to make a universal device adaptive to all people. These solutions are usually the combination of pads, belts, cushions that are interconnected with flexible/adjustable materials. They are visually, physically, psychologically uncomfortable and not suitable for usage outside the hospital. In the present invention, ECG electrodes are embedded in a garment, making ECG electrodes wearable, comfortable and aesthetic like any other clothing.

The present invention has advantage over the other ECG wearable garments, none of which proposed a solution to enable untrained people to administrate clinical ECG.

The present invention can facilitate ordinary people to self-administrate ECG with the help of several selected bony landmarks, which can be recognized easily by non-professionals.

The basic structure of the present invention is a garment with labels for several selected bony landmarks that are most helpful for electrodes positioning, including clavicle, superior border of manubrium sterni, midsternal line, inferior border of corpus sterni, midaxillary line and inferior border of thoracic cage at midaxillary line. The labels are fixed at corresponding position of the bony landmarks on the garment, which is a strong reference for the position of electrodes. All the embodiments of the present invention are developed from this basic structure, the details of which will not be repeated in each embodiment.

Customization to the garment of the present invention is necessary to ensure the accuracy of the electrode positions and their abutment to the skin. The garment will be available in different sizes to adapt to various torso height and avoid the unstable axial textile stretch. The garment of the present invention can be tailored for better adaptation to various body shapes, e.g. female breasts. There will also be customized versions in body landmark position to reflect to the variations in thoracic cage.

Consumers should choose the correct garment most suitable for their body. When the bony landmark labels align with the corresponding bony landmarks, the garment is ‘correctly’ worn. The elasticity of the textile can offset the impact of body shape on the positions of electrodes to ensure the accuracy of the position of electrodes.

Basing on the basic structure, the first embodiment of the present invention is a garment with aperture at the target electrode positions. After putting on the garment ‘correctly’, the subject can find the right electrode position as indicated by the apertures. The subject can put on the garment and connect ECG electrodes to the skin through the apertures. This fundamental solution features low-cost, convenient, wash-safe, and compatible with almost any ECG devices. However, the apertures only indicate the positions of the electrodes. The subjects still have chance to make mistake by putting an electrode in other electrode's position.

As an alternative to the first embodiment, if the customers have special body shape or have concern with the accuracy of the electrode positions, the bony landmark labels and electrodes apertures/labels are provided separately with the garment. With the help of professionals, the bony landmark labels and electrodes apertures/labels can be fixed to the right position on the garment.

Basing on the basic structure, in another embodiment of the present invention, the garment contains conductive electrode connectors, ECG lead wires and an external ECG device connector. Reusable or disposable electrodes can be snapped or adhered to the conductive electrode connector. The wires start from electrode connectors, go within the textile or interlayer of the garment, aggregate in a signal bus and end in an external ECG device connector. This kind of garment is ready for use with attached electrodes. This design is more user-friendly, however costlier, less wash-safe with embedded wires and connector. It is only compatible with devices that can adapt to the connector.

Basing on the basic structure, in yet another embodiment, the electrodes, wires, external ECG device connector are all integrated on the garment. The electrodes are fixed on the right position. The wires start from the electrodes, go within the textile or interlayer of the garment, aggregate in a signal bus and end in an external ECG device connector. The garment is out-of-box ready for use.

Basing on the basic structure, in another embodiment, the positions of integrated electrode and the bony landmarks are customizable. The labels for bony landmarks are provided separately as accessories of the garment. The external ECG device connector and ECG lead wires are fixed on the garment. The wires start from the connector, go within the textile or interlayer of the garment and end in ECG electrodes.

At the electrode end of the wire, a small segment of wire goes out of the fabric or interlayer from the inside surface of the garment and is free to move. Each electrode is fixed on the corresponding wire instead of being fixed on the garment. The electrode and the free wire end can move within a limited scope for minor adjustment of the electrode position. The subject can fix the electrodes and the bony landmark labels on the garment at the accurate position with the help of professionals.

The present invention is by no means an ultimate solution for all the problems associated with ECG electrode positioning and fixing. In occasions when the anatomical structure of the chest is not uniformly convex, or when protrusions on the body (as at the pectorals or breasts) create concavities (as at the sternum), the elastic material might not be able to exert enough pressure between two convex portions of the body, causing poor or nonexistent electrode abutment. Though the tailoring solution to fit female breast and the customization design (claim 4) might be helpful for some of these special conditions, there are small portion of subjects that cannot get a satisfying ECG administration from the present invention. The present invention aims to provide a practical solution that can meet the requirements of most of the people with regular body shape. The vision is to make ECG device a common daily wearable acceptable by the ‘healthy’ people. There might be consequent enhancements to the present invention to make it applicable to special conditions mentioned above.

### BRIEF DESCRIPTION OF DRAWINGS

FIG. 1. Front view of the elastic garment with labels to indicate selected bony landmarks. There are apertures at the position of the electrodes, each marked with a label with unique color/shape/code.

FIG. 2. Front view of the elastic garment. The electrode apertures, labels for bony landmarks, labels for electrodes are provided separately as accessories.

FIG. 3. Front view of the elastic garment with embedded conductive electrode connectors, ECG lead wires and an external ECG devices connector.

FIG. 4. Front view of the elastic garment with integrated ECG electrodes, wires and external ECG device connector.

FIG. 5. Front view of the elastic garment with integrated ECG electrodes, wires and external ECG device connector. The labels for body landmarks are provided separately as accessories. The electrodes position is not fixed and can be slightly adjusted.

FIG. 6. Three-quarters left side perspective view of one embodiment of the present invention when worn.

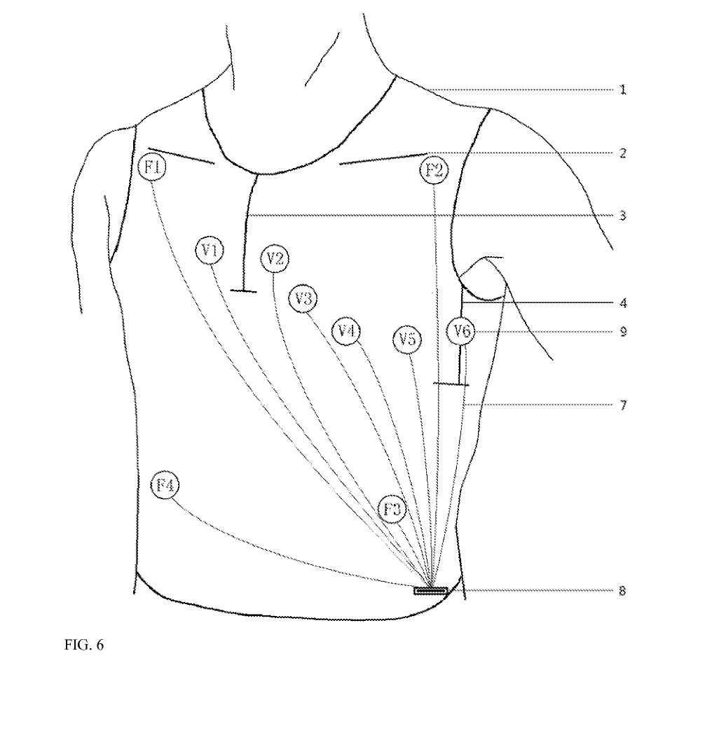

### DETAILED DESCRIPTION OF THE INVENTION

The present invention relates to an ECG electrode wearable, and more particularly to an elastic ECG electrode wearable garment that enables accurate positioning and fixing of ECG electrodes.

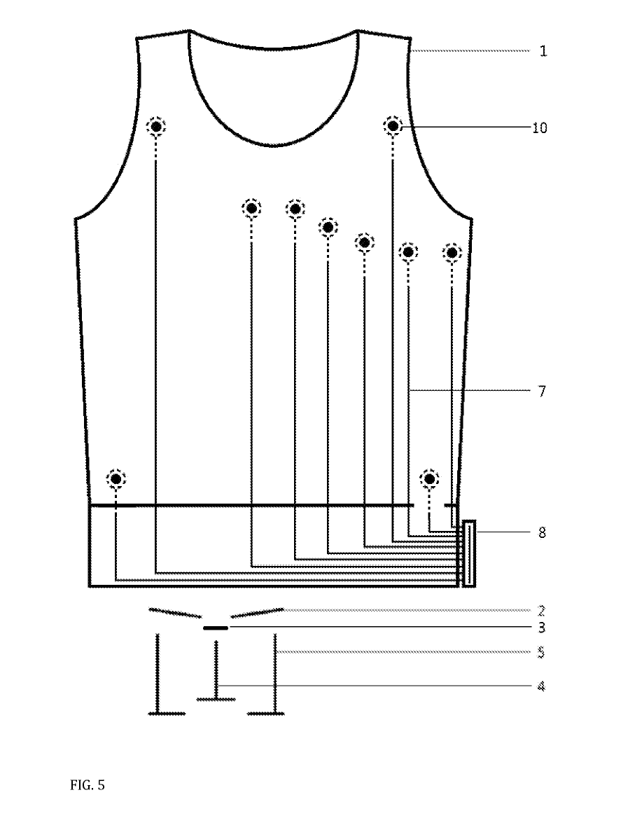

Figures show different aspects of preferred embodiments of the invention. FIG. 5. illustrates the perspective view of the present invention.

The basic structure of the invention consists of an elastic garment (1) to be worn about the subjects' body and labels on the outside surface of the garment to indicate the position of selected bony landmarks. The garment (1) is made of any elastic textile commonly used and supports a multiplicity of ECG electrodes, the arrangement of which includes but is not limited to standard 12-lead ECG. Actually the electrodes can be fixed at any position on the garment by sampling requirements. The labels for bony landmarks include label for clavicle (2), label for superior border of manubrium sterni (3), label for midsternal line and inferior border of corpus sterni (4), label for midaxillary line and inferior border of thoracic cage at midaxillary line (5). The labels can be visible and/or tangible, which are clipped, adhered, stitched or melt on the garment at the corresponding position to the bony landmarks.

The garment (1) can be tailored or designed for better adaptation to body shapes and ensure electrode contact with the skin, which is especially important for positioning and fixing electrodes around female breasts. For example, the garment can comprise an inner brassiere layer to better adapt to the shape of female breasts. The brassiere can lift the breast, ensure electrode abutment to the skin and avoid electrode dislocation.

The garment (1) will be available in different sizes to adapt to different torso height. Though the garment is elastic, the stretch of the textile along the long axis of the torso does not have a firm anchor to prevent the textile from contraction. A nice fit in size is necessary to avoid the unstable axial textile contract which is easily to cause electrode displacement. Different chest/bust size will also be available for different body build.

The garment (1) can be presented in any form of clothes that can cover at least precordial area, including brassiere, top, tank top, base layer, pajama, shirt, jersey, sweater, sweatshirt, pullover, vest, hoodie, jacket, coat, overall, bra, halter, bikini, jumpsuit, romper, overall, coverall, other lingerie or the combination of these styles.

Different form of the garment (1) has different supporting ability to electrode position.

The forms of the garment that can cover limbs can support standard 12-lead applications, while the forms that cannot cover limbs can only support alternative applications like Mason-liker lead positions.

The labels for clavicle (2) and the labels for superior border of manubrium sterni (3) have equivalent function of ensuring that the garment is correctly worn at the upper section of the torso. The garment can have either labels for clavicle (2) or the labels for superior border of manubrium sterni (3).

Consumers should choose the garment which is most suitable to their body. When the bony landmark labels align with the corresponding bony landmark, the garment is correctly worn up. The elasticity of the textile will exert pressure surrounding the torso to ensure skin abutment and avoid electrode dislocation. The proportional expansion of the textile can offset the impact of chest/bust size on the positions of electrodes to ensure the accuracy of the position of electrodes.

Based on this basic structure, several embodiments were provided.

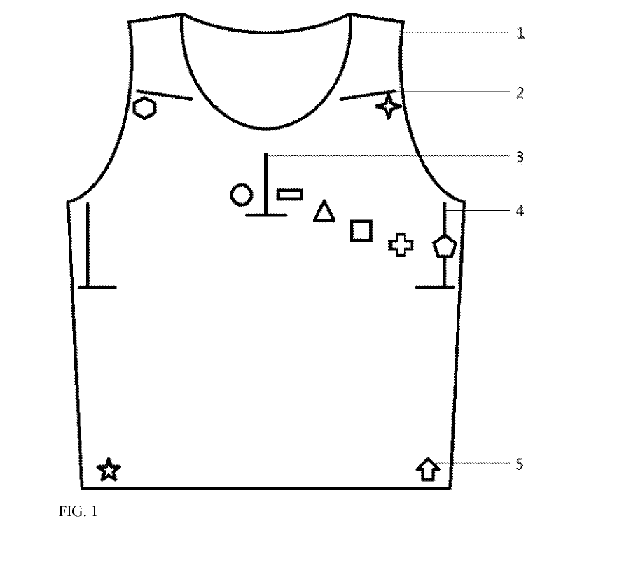

FIG. 1 illustrates the simplest embodiment of the present invention. At the electrode positions on garment (1), there are electrode apertures with unique label differentiated by color, shape and code (6). The positions of the electrode apertures can follow international standard 12-lead or any other applications required for clinical purpose.

When the subject wears the garment correctly, the apertures can indicate the right position of the electrode. User can connect electrodes to the skin through the aperture.

The wires should go outside the garment to ensure comfortability. This is the fundamental solution of the present invention, featuring low cost, wash-safe and good compatibility with almost all of the current ECG devices.

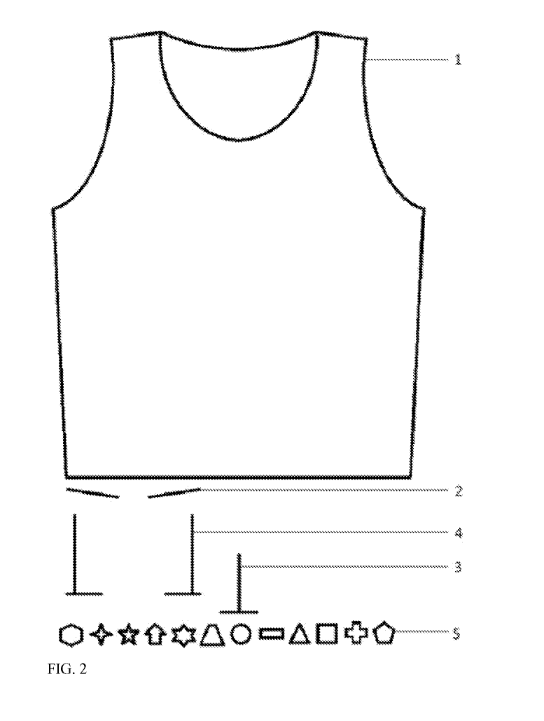

FIG. 2 illustrates an alternative solution of FIG. 1 of the present invention. For individuals with special body shape or with concern on the accuracy of the position of the indicators, the position of bony landmarks and the position of the electrode apertures can be customized. The labels for bony landmarks (2 thru 5), the electrode apertures with unique label differentiated by color, shape and code (6) are provided separately as accessories of the garment (1). The electrode aperture is actually a (circle) frame before being fixed to the garment. User should remove the textiles surrounded by the frame after it has been fixed to the garment. User can put on the elastic garment (1) and ask professionals to help locate the position of the bony landmark (2 thru 5) and the electrode apertures with unique label differentiated by color, shape and code (6). Then user can clip, adhere, stitch or melt the labels for bony landmarks (2 thru 5), the electrode apertures with unique label differentiated by color, shape and code (6) on the right position of the garment.

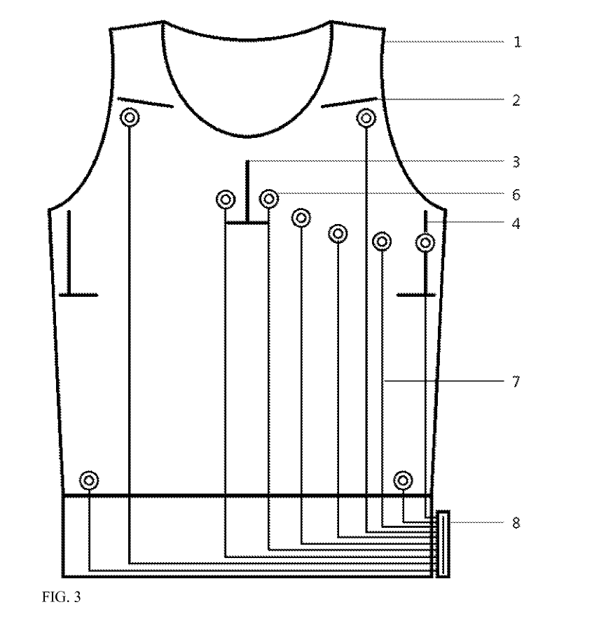

Despite all the advantages of the embodiments of FIG. 1. and FIG. 2. like wash-safe, good compatibility, they cannot definitely prevent wrong electrode connection. The subject might put the electrode in the wrong aperture. A better way to avoid electrode mis-connection is presented in FIG. 3. In this design, conductive electrode connectors (6) are fixed on the garment by the standard 12-lead application or any other clinically required applications. Reusable or disposable electrodes can be adhered or snapped onto the conductive electrode connectors (6). The contact area between the electrode connector and the electrode is ‘conductive’ and can conduct ECG signals from ECG electrodes to the lead wire. ECG lead wires (7) start from the conductive electrode connectors (6), go within the textile or the interlayer of the garment, aggregate into a signal bus and end in an external ECG device connector (8). The external device connector is fixed on the garment. This device is ready for use after electrodes have been fixed to conductive electrode connectors (6) on the inner surface of the garment.

The electrodes and wires can be any feasible solution available in the market providing that they can ensure high-quality ECG signals transmission.

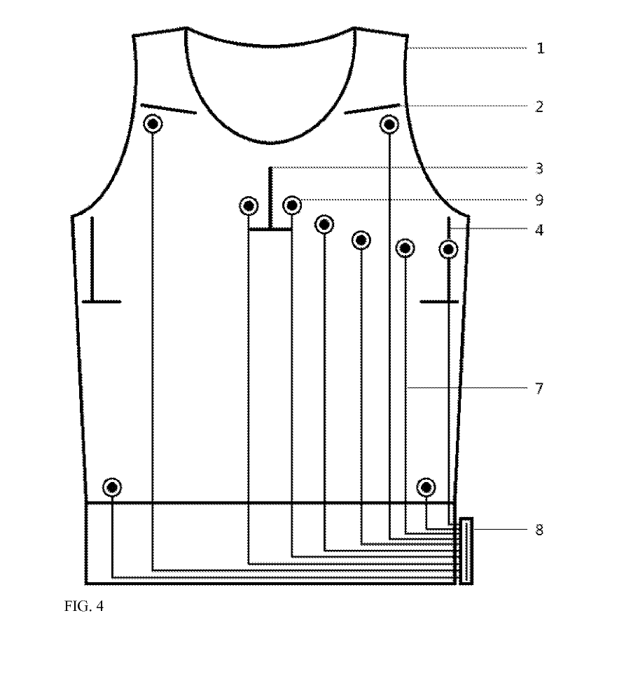

Another embodiment of the present invention is illustrated in FIG. 4. In this solution, ECG electrodes (9), ECG lead wires (7) and an external ECG device connector (8) are integrated on the garment. The electrodes (9) are fixed on the garment by the standard 12-lead application or any other clinically required applications. ECG lead wires (7) starts from the electrodes (9), go within the textile or interlayer of the garment, aggregate into a bus and end in an external ECG device connector (8). This device is out-of-box ready for use. The electrodes and wires can be any feasible solution available in the market providing that they can ensure high-quality ECG signals transmission.

Another embodiment of the present invention is illustrated in FIG. 5. In this embodiment, ECG electrodes (9), ECG lead wires (7) and an external ECG device connector (8) are integrated on the garment, and the positions of the ECG electrodes (9) can be slightly adjusted. The ECG lead wires (7) start from the external ECG device connector (8), go within the textile or interlayer of the garment (1), come out of the textile at the position of the corresponding electrodes in the inner surface of the garment, go free for a short distance before connecting with the ECG electrodes (9).

The electrodes (9) are free to move within a limited scope for accurate adjustment of the electrode position. The labels for bony landmarks (2 thru 5) are provided separately as accessories of the garment (1). The subject can clip, adhere, stitch or melt the ECG electrodes (9) and the labels for bony landmarks (2 thru 5) at the right position with the help of professionals.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

1. An elastic wearable garment that helps positioning and fixing ECG electrodes, the garment comprising: an elastic garment to be worn about the subjects' body and having firm contact with the subjects' torso; visible and/or tangible labels on the outside surface of the garment to indicate the position of bony landmarks including labels for clavicle, label for superior border of manubrium sterni, label for midsternal line and inferior border of corpus sterni, labels for midaxillary line and inferior border of thoracic cage at midaxillary line, which are fixed at the corresponding positions to the body landmarks on the garment.

2. The garment of claim 1, wherein the labels for clavicle are optional.

3. The garment of claim 1, wherein the label for superior border of manubrium sterni is optional.

4. The garment of claim 1, 2 or 3, wherein the labels for bony landmarks are not fixed on the said elastic garment but provided separately.

5. The garment of claim 1, 2 or 3, wherein the garment further comprises apertures opened at the positions of the ECG electrodes, the apertures each corresponding to one ECG electrode and having one attached label with unique color, shape and code to indicate the corresponding electrode, the arrangement of said apertures following standard 12-lead method, mason-liker leads method, or any other clinically accepted method.

6. The garment of claim 4, wherein the garment further comprises electrode apertures in the form of frame or ring, the apertures not being fixed on the garment but provided separately, each said aperture further comprising label with unique color, shape and code to indicate the corresponding electrode.

7. The garment of claim 1, 2 or 3, wherein the garment further comprising: conductive electrode connectors fixed at the position of the ECG electrodes on the inside surface of the garment, each said conductive electrode connectors corresponding to one ECG electrode and connecting to an ECG lead wire, the position of the said conductive electrode connectors following standard 12-lead method, mason-liker leads method, or any other clinically accepted method; ECG lead wires starting from conductive electrode connectors, woven in the textile or fixed within the interlayer of the garment, aggregating in an signal bus and terminating in an external ECG device connector; the external ECG device connector that can be connected with external ECG devices.

8. The garment of claim 1, 2 or 3, wherein the garment further comprising: ECG electrodes fixed on the inside surface of the garment, each said ECG electrode connecting to an ECG lead wire, the positions of the said ECG electrodes following standard 12-lead method, mason-liker leads method, or any other clinically accepted method; ECG lead wires connecting ECG electrodes and external ECG device connector, said ECG lead wires woven in the textile or fixed within the interlayer of the garment; an external ECG device connector for collecting ECG signals, connecting to external ECG processing devices and transmitting ECG signals to the external ECG processing devices.

9. The garment of claim 4, wherein the garment further comprising: ECG electrodes connected to ECG wires and not fixed on the garment; ECG lead wires connecting ECG electrodes and external ECG device connector, a short length of the electrode end of each said ECG lead wire being free and not fixed on the garment, the rest of the said ECG lead wires going in to the textile at the positions of ECG electrodes from the inside surface of the garment, woven in the textile or fixed within the interlayer of the garment, the positions of electrodes following standard 12-lead method, mason-liker leads method, or any other clinically accepted method; an external ECG device connector for collecting ECG signals, connecting to external ECG processing devices and transmitting ECG signals to the external ECG processing devices.

10. The garment of above claim 1 thru claim 9, wherein the garment can be made into following styles including brassiere, top, tank top, base layer, pajama, shirt, jersey, sweater, sweatshirt, pullover, vest, hoodie, jacket, coat, overall, bra, halter, bikini, jumpsuit, romper, other lingerie or the combination of these styles.

11. The garment of above claim 1 thru claim 9, wherein the garment can be any style of clothes that can cover at least precordial area.
